(19) United States
(12) Patent Application Publication
(10) Pub. No.: US 2025/0387445 A1
(43) Pub. Date: Dec. 25, 2025

(54) MELATONIN-ENRICHED COMPOSITION OF PISTACIA VERA (PISTACHIO) AND A METHOD OF PREPARATION THEREOF

(71) Applicant: Network Nutrition Pty Limited, Kiama NSW (AU)

(72) Inventor: Ryan Gorman, Kiama NSW (AU)

(21) Appl. No.: 19/249,155

(22) Filed: Jun. 25, 2025

Related U.S. Application Data
(60) Provisional application No. 63/663,988, filed on Jun. 25, 2024.

(51) Int. Cl. A61K 36/22; A61K 31/047; A61K 31/05; A61K 31/198; A61K 31/4045; A61K 31/405; A61P 25/20

(52) U.S. Cl. CPC A61K 36/22; A61K 31/047; A61K 31/05; A61K 31/198; A61K 31/4045; A61K 31/405; A61P 25/20; A61K 2236/19; A61K 2236/33; A61K 2236/37

(57) ABSTRACT

Provided herein are methods and compositions for a melatonin-enriched composition of Pistacia vera (Pistachio).

### BACKGROUND

The invention relates to the selection, preparation and extraction of Pistachio seed kernel to achieve a standardized level of natural-source melatonin.

Melatonin is widely established as a dietary supplement ingredient and is primarily employed for the purpose of supporting and regulating healthy sleep patterns, overcoming jetlag, and managing mild sleep disturbances.

Melatonin was once produced commercially from bovine and porcine pineal gland and was thus animal derived. With the escalation of concerns around animal origin, particularly with the arrival of viruses such as mad cow and foot-and-mouth disease, alternative sources of melatonin were sought. The majority of melatonin dietary supplements available commercially utilize synthetically produced melatonin.

As more populations transition to sustainable, plant-based diets, there is a growing need to provide ingredients such as melatonin from plant-based sources. It is also understood that synthetically derived melatonin is subject to significant quality variation, both with respect to the ingredient itself and the dietary supplement/food products that contain it.

Sleep is a finely tuned process driven by intricate neurochemical interactions within the brain's sleep promoting and arousal centres. At the heart of this regulation lies melatonin, influencing various body systems and physiological processes that in turn induce immediate physiological responses such as fatigue, and long-term health outcomes due to its powerful antioxidant properties. Melatonin exerts this influence by activating highly sensitive receptors MT1 and MT2 in response to changes in the light-dark cycles directly effecting the synchronization of circadian rhythm, sleep-wake cycles and the duration and stages of sleep experienced. While both receptors are activated by melatonin, they have been reported to elicit distinct physiological responses due to the unique expression patterns, signaling pathways and pharmacological properties across various body organs. Activation of MT1 receptors reduces nerve cell activity in the suprachiasmatic nucleus (SCN), promoting sleep initiation, while MT2 receptor activation regulates the synchronization of the circadian rhythm in response to environmental clues, such as the light-dark cycles. The synergistic action of these receptors in regulating sleep timing, duration and circadian rhythms underscores their potential as valuable pharmacological targets aimed at optimizing sleep management and quality.

Clinical studies have extensively explored the administration routes of melatonin, primarily focusing on oral and intravenous methods in healthy volunteers. However, the findings regarding its pharmacokinetics have shown inconsistency.

Studies have revealed that oral melatonin follows first-order kinetics, with bioavailability averaging around 15%, although it can vary significantly among individuals. Various factors, including age, caffeine consumption, smoking, oral contraceptive use, feeding status, and concurrent medications like fluvoxamine, influence its pharmacokinetics. Despite this, melatonin appears to maintain a consistent half-life of approximately 45 minutes, with the peak concentration in the bloodstream reportedly occurring approximately 50 minutes after oral dosage.

The present invention attempts to solve these problems, as well as others.

### SUMMARY OF THE INVENTION

Provided herein are methods and compositions for a melatonin-enriched composition of Pistacia vera (Pistachio).

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1A is a flow chart showing the method for preparing a melatonin-enriched extract of Pistacia vera (Pistachio) seed/kernel, according to one embodiment.

FIG. 1B is a flow chart showing the method for preparing a melatonin-enriched extract of Pistacia vera (Pistachio) seed/kernel, according to one embodiment.

FIG. 2A is a graph showing an example chromatogram of standard; FIG. 2B is a graph showing an example chromatogram of sample; and FIG. 2C is a graph showing an example chromatogram of blank.

FIG. 3A is a graph showing the melatonin absorption over 2-hours and FIG. 3B is a graph showing the melatonin absorption over 8-hours. FIGS. 3A-3B is created with any negative value recorded as zero, and as a group average for each specific time point. Data presented is mean±SEM.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and manufacturing arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### DESCRIPTION OF EMBODIMENTS

Generally speaking, the method of preparing melatonin from Pistachio provides a more effective dose/serving of melatonin than synthetic source in regulating sleep in a readily accessible form that can be consumed in dietary supplement or food products. The starting material of Pistachio is widely consumed around the world and has good acceptance as a healthy food. It is however not widely understood that Pistachio is a rich source of melatonin, and this method achieves enrichment of that starting material to maximize the content of melatonin in the final extract, such that an appropriate quantity of the melatonin composition can be consumed in either dietary supplement or food format to provide a meaningful effect. This is the first instance of melatonin being enriched from Pistacia vera (Pistachio) and presented in a form appropriate for use in pharmaceutical and/or food presentation.

Certain cultivars of pistachios containing high levels of melatonin, exceeding 12,000 pg/g. Additionally, amongst other nuts, pistachios include significantly higher levels of Phyto-melatonin, with some varieties containing up to 223 ug/g. These findings highlight pistachio-derived melatonin foods as a potential therapeutic option. The Examples below assess the efficacy of a natural, food derived melatonin gummy compared to a synthetic melatonin gummy for melatonin absorption and sleep quality.

In one embodiment, the melatonin composition is bound within a complex herbal matrices, including such complementary compounds as arginine, tryptophan, lutein, resveratrol, and with a high ORAC antioxidant capacity. The melatonin composition bound with complex herbal matrices increases the bioavailability, absorption, and effectiveness of melatonin when compared to synthetic melatonin. The melatonin extract is biologically active on MT1 and MT2 receptors in terms of binding and signal transduction. The melatonin extract increases the activity of the endogenous melatonin as well as increasing exogeneous melatonin, thus being a synergistic effect on treating sleep conditions. In one embodiment, the treatment of a sleep condition comprising administering low amounts of the melatonin extract in the amounts of about 0.03 g to about 0.5 g of the melatonin extract, which is equivalent to about 300 mcg to about 5 mg of natural melatonin as to trigger a physiological effect in humans.

The melatonin composition comprises a melatonin-enriched extract of Pistacia vera (Pistachio) seed/kernel to address the need for a plant-based source of melatonin that can provide a meaningful and effective dose of melatonin in a convenient dosage/serving.

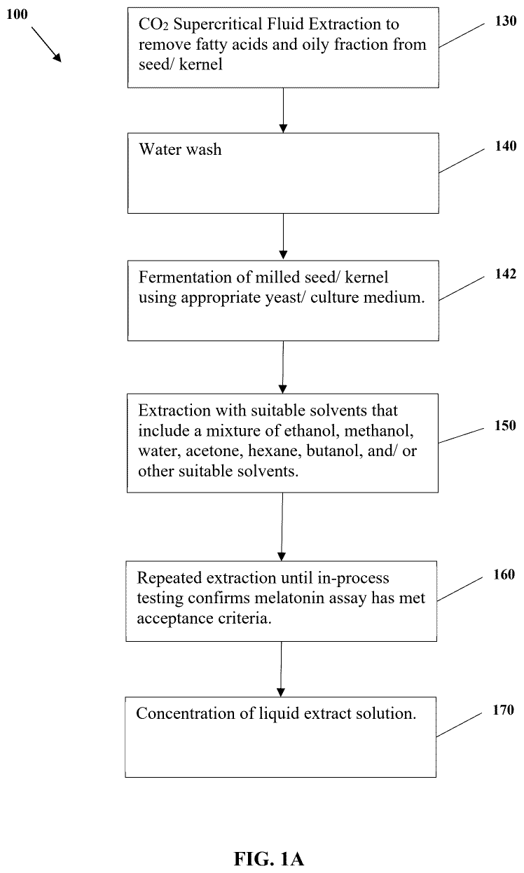

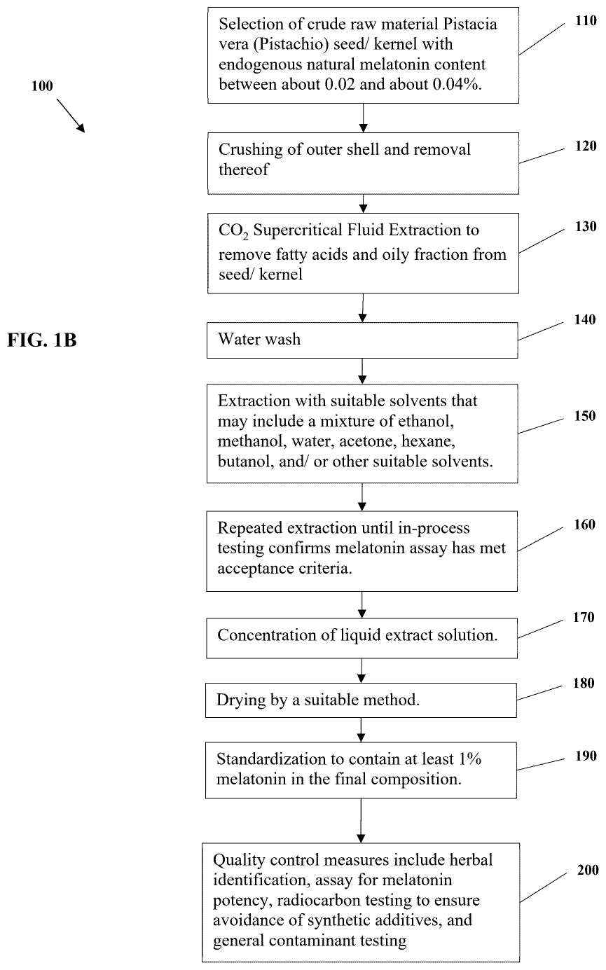

As shown in FIG. 1A, the method for preparing a melatonin-enriched extract of Pistacia

vera (Pistachio) seed/kernel 100 comprises the step of 130 of CO₂ Supercritical Fluid Extracting to remove fatty acids and oily fraction from seed/kernel to generate a melatonin extract; step 140 of washing the melatonin extract with water; alternatively, step 142 fermentation of milled seed/kernel using a yeast/culture medium; step 150 of extracting the melatonin with suitable solvents that may include a mixture of ethanol, methanol, water, acetone, hexane, butanol, and/or other suitable solvents; step 160 of repeated extracting until in-process testing confirms melatonin assay has met acceptance criteria; step 170 of concentrating the liquid extract solution. In one embodiment, the acceptance criteria is Not less than (NLT) about 1.0% as determined by a High Performance Liquid Chromatography (HPLC) assay.

As shown in FIG. 1B, the method for preparing a melatonin-enriched extract of Pistacia vera (Pistachio) seed/kernel 100 comprises the step of 110 including selecting crude raw material Pistacia vera (Pistachio) seed/kernel with endogenous natural melatonin content between about 0.02 and about 0.04%; step 120 including crushing of outer shell and removal thereof; step 130 of CO₂ Supercritical Fluid Extracting to remove fatty acids and oily fraction from seed/kernel; step 140 of washing with water; step 150 of extracting with suitable solvents that may include a mixture of ethanol, methanol, water, acetone, hexane, butanol, and/or other suitable solvents; step 160 of repeated extracting until in-process testing confirms melatonin assay has met acceptance criteria; step 170 of concentrating the liquid extract solution; step 180 of drying by a suitable method; step 190 of standardizing to contain at least 1% melatonin in the final composition; and step 200 of applying quality control measures include herbal identification, assay for melatonin potency, radiocarbon testing to ensure avoidance of synthetic additives, and general contaminant testing (i.e.; Solvent residues, pesticide residues, aflatoxins, microbiology, heavy metals, pyrrolizidine alkaloids, polycyclic aromatic hydrocarbons. Standardization may be achieved either by sub-lot blending or through the use of an appropriate carrier/excipient. In one embodiment, the melatonin extract is about a 16:1 to about a 20:1 native extract ratio, in another embodiment, the melatonin extract is about a 15:1 ratio. In one embodiment, the solvent is ethanol and water at a ratio of about 30:70.

An overview of the process to achieve the present disclosure is provided in Manufacturing Flow Chart bearing document code NNPVE151-MFC (valid edition).

### Melatonin Structure

The chemical structure for melatonin is described by United States National Institutes of Health National Library of Medicine I https://pubchem.ncbi.nlm.nih.gov/compound/Melatonin.

The chemical structure of synthetic melatonin is the same structure as found in the present disclosure.

### Dosing

The melatonin composition may be in the form of capsules that consumers can take orally. The melatonin composition is free from any contaminants, including foreign material, pesticides, bacterial pathogens, molds, residual solvents, heavy metals. A certificate of analysis (COA) documented the analytical tests for melatonin composition.

The methods described herein may comprise administering daily, or every other day, or once a week, an effective dose of the Melatonin composition. In an embodiment, the Melatonin composition is administered daily for 1-2 weeks, 1-3 weeks, 1-12 week, 4-8 weeks, 8-12 weeks, 1-12 weeks, 4-12 weeks, 8-12 weeks, or 12-15 weeks, or more, or for another period of time according to the present invention.

The Melatonin composition of the present invention may be administered in combination with one or more nutraceutically acceptable carriers and/or other excipients. The active ingredients in such formulations may comprise from 1% by weight to 99% by weight, or alternatively, 0.1% by weight to 99.9% by weight. “Nutraceutically acceptable carriers and/or other excipients” means any carrier, diluent or excipient that is compatible with the other ingredients of the Melatonin composition and not deleterious to the user. In accordance with one embodiment, suitable nutraceutically acceptable carriers can include oils, plant-based oils, Medium-chain triglyceride (MCT) oil, coconut oil, palm kernel oil, Hemp seed oil, Olive oil, Avocado oil, and combinations thereof. Carriers may include dicalcium phosphate, rice flour, maltodextrin, microcrystalline cellulose, etc. Other excipients may include binders (such as hydroxypropyl cellulose, Hypromellose, various gums, etc.), flow agents/anticaking agents (such as silicon dioxide, etc.), disintegrants (such as croscarmellose sodium, etc.), lubricants/mold release agents (such as magnesium stearate, calcium laurate, etc.), flavorings/sweeteners, coloring, or other excipients as needed for the dosage form.

In certain embodiments, the dosage form is formulated as granules, pellets, micro particles, tablet, hard shell capsules, suspended in a liquid, suspended in a syrup or enema. In certain embodiments, the dosage form is formulated for oral or mucosal delivery. In certain embodiments, the dosage form is formulated as or in a lozenge, candy, gummy, chocolate or cookie. In certain embodiments, the tablet or pellets are an immediate release or slow or controlled release dosage forms. In certain embodiments, the tablet is enteric coated or is a melt or dissolved in the mouth or is muco-adhesive dosage form.

In certain embodiments, the unit dosage form which is a unit particle, such as tablet, capsule, granules, pellets, micro-particles and film, are enteric coated or coated with a colonic coat that protect the unit dose from being decomposed at the acidic gastric pH and swells in time manner of pH-controlled manner or both, to release the Melatonin composition at the distal intestine.

In certain embodiments, the Melatonin composition is formulated in a semi solid or liquid dosage form such as cream, lotion, ointment, dispersion, suspension, gel, foam, spray, syrup, liquid, eye drops, ear drops, enema or an oral dosage form or a topical dosage form or a local ophthalmic or optic or oral cavity or vaginal or rectal or uterine dosage form. Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose for in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

In certain embodiments, any one of the compositions described above, or any one of the dosage forms described above, is for use in a method of treating insomnia or sleep disorders.

Preferred dosage forms include, but are not limited to, any liquid or semi solid or solid dosage form. The Melatonin composition may be formulated in a medicament by preparing a topical or mucosal or oral delivery system. The topical delivery system may be in the form of eye drops, a suspension, ointment, cream, foam, spray, topical patch. The oral delivery system may be a tablet or capsule or soft capsule or sachet or granules or a syrup. The mucosal delivery system may be a gel, pessary, enema, douche, wash, foam, mucoadhesive gel or tablet for immediate or for slow or controlled release. The vehicle may comprise any acceptable solvent and inactive ingredients as well as preservatives, anti-oxidants and coloring agents. The delivery form may be single dose or multiple dose as well as micro particle granulate nanoparticle microcapsule liposome micelle, and the like as known in the art of pharmaceutical, cosmetic, veterinary medicine and art of formulation. Further details of suitable dosage forms may be obtained from any standard reference work in this field, including, for example: Remington's Pharmaceutical Sciences, Mack Publishing Co, Easton, Pa, USA (1980).

Thus, in some embodiments of the present invention, the source of Melatonin composition further comprises one or more excipients selected from the group consisting of solvents, stabilizers, suspending agents, emulsifiers, release modifying, targeting and viscosity agents and combinations thereof. In one embodiment, the excipient is about 10% to about 25% Maltodextrin.

### Method of Treatment

The term “treating” or “increasing” are used interchangeably herein. These terms can refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “synergistic” as used herein refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-anxiety; ingredient B is pro-anxiety; when A and B are combined, said combination produces an anti-anxiety effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

The term “composition” as used herein refers to a composition which is pharmaceutically acceptable and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The formulation includes configurational isomers (such as cis and trans isomers) and all optical isomers (such as enantiomers) Isomers and diastereomers), racemic, diastereoisomers and other mixtures of these isomers, as well as solvates, hydrates, isomorphs, polymorphs, tautomers, Ester, salt forms and prodrugs. The term “prodrug” refers to a compound that is a drug precursor, which releases the drug in vivo through some chemical or physiological processes after administration (for example, the prodrug is transformed into the desired drug form when it reaches physiological pH or through the action of enzymes).). Exemplary prodrugs release the corresponding free acid upon cleavage, and the hydrolyzable ester-forming residues of the compounds of the present invention.

In the present disclosure, an “effective amount” or an “effective dose” of melatonin extract, or composition refers to an amount of melatonin extract from Pistachio that, once administered to a subject, will reach the subject's bloodstream, intestine, and/or bodily tissues. In one embodiment, the method comprises administering or taking the melatonin

composition in a single formulation to increase synergy between the exogenous melatonin and endogenous melatonin. The method comprises combining the melatonin composition with the herbal matrices into a formulation to increase the synergy in treating a sleep condition or increasing sleep efficacy.

The methods described herein may comprise administering daily, or every other day, or once a week, an effective dose of the melatonin composition with the herbal matrices. In an embodiment, the melatonin composition with the herbal matrices is administered daily for 1-2 weeks, 1-3 weeks, 1-12 week, 4-8 weeks, 8-12 weeks, 1-12 weeks, 4-12 weeks, 8-12 weeks, or 12-15 weeks, or more, or for another period of time according to the present invention. The method of treating a sleep disorder comprises administering an effective amount

of the melatonin extract from Pistachio and an effective amount of the herbal matrices. “Sleep disorder” is any condition where a subject has difficulty falling asleep and difficulty staying asleep such as insomnia, and includes conditions related to difficulty falling asleep due to anxiety, stress, and depression; difficulty sleeping resulting in poor concentration and focus; difficulty falling asleep due to age-related memory loss, dementia, or other cognitive disorder; difficulty falling asleep due to substance abuse, mental disorders, breathing disorders, or by other sleep disorders such as periodic limb movement; difficulty falling asleep or staying asleep due to other sleep related problems such as poor sleep hygiene, poor sleep hygiene includes consumption of beverages with alcohol or caffeine, eating large meals, or engaging in physically or mentally stimulating activity shortly before bed time; poor sleep hygiene includes a highly variable bed time, or inadequate temperature, poor ventilation, noise or light within the sleep environment; including narcolepsy, and Rapid Eye Movement (REM) sleep behavior disorder; disorders associated with neurodegenerative diseases such as Alzheimer's disease, Parkinson's disease, and multiple sclerosis; disrupted REM sleep disorder associated with drug withdrawal, especially alcohol or sedative-hypnotic withdrawal; and disrupted circadian rhythm associated with sleep apnea, shift work and jet lag.

### Complex Herbal Matrices

The complex herbal matrices include complementary compounds including arginine, tryptophan, lutein, resveratrol, and with a high ORAC antioxidant capacity.

Arginine is the amino acid with the formula (H₂N)(HN)CN(H)(CH₂)₃CH(NH₂)CO₂H. L-arginine is recognized as safe (GRAS-status) at intakes of up to 20 grams per day. L-arginine (0.15-0.60 micromol), a nitric oxide precursor, regulates sleep variables such as increased slow wave sleep and reduced waking.

Tryptophan is an α-amino acid that is used in the biosynthesis of proteins. Tryptophan contains an α-amino group, an a-carboxylic acid group, and a side chain indole, making it a polar molecule with a non-polar aromatic beta carbon substituent. Tryptophan is also a precursor to the neurotransmitter serotonin, the hormone melatonin, and vitamin B3. Tryptophan contributes to good sleep because the body uses it to make serotonin, a neurotransmitter. Serotonin helps regulate sleep and is used to create melatonin, a sleep-promoting hormone.

Lutein is a xanthophyll and one of 600 known naturally occurring carotenoids. Lutein has been shown to reduce short sleep durations, i.e. sleeping less than 7 h/day. Lutein is isomeric with zeaxanthin, differing only in the placement of one double bond. Lutein and zeaxanthin can be interconverted in the body through an intermediate called meso-zeaxanthin. Pistachios contain 1,205 mcg of lutein per 100 g.

Resveratrol (3,5,4′-trihydroxy-trans-stilbene) is a stilbenoid, a type of natural phenol or polyphenol and a phytoalexin. Resveratrol exists as two geometric isomers: cis-(Z) and trans-(E), with the trans-isomer shown in the top image. Resveratrol exists conjugated to glucose. Resveratrol has been known to induce sleep and reduce active-awake time and paradoxical sleep patterns. Sleep observations and brain wave measurements suggested that Resveratrol offered a sufficient improvement in non-REM sleep, which is believed to be the most important aspect of sleeping properly.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: HPLC Analytical Method

Purpose: To describe the procedure used to determine the content of Melatonin in Pistacia vera by UPLC-MS.

TABLE 1a

| Technical Data: | | | |
|---|---|---|---|
| Instrument | UPLC fitted with ESI-MS detectors in sequence | | |
| Solvents | 5% Methanol (aqueous) | | |
| Column | Shimadzu Shim-pack Scepter HD-C18-80, 100 mm × 2.1 mm, 1.9 | | |
| | μm (Part No 227-31026-05) or equivalent. | | |
| Pre-column | N/A | | |
| Column temperature | 40° C. | | |
| Mobile Phase | Solution A: 0.1% v/v Formic acid (LCMS | | |
| | grade) Solution B: Acetonitrile (LCMS grade) | | |
| Gradient | Time (min) | Solution A (%) | Solution B |
| | (%) 0−5 | 95 | 5 |
| | 5−10 | 95−60 | 5−40 |
| | 10−15 | 60−0 | 40−100 |
| | 15−20 | 0 | 100 |
| | 20−25 | 95 | 5 |

| | |
|---|---|
| Flow rate | 0.5 mL/min |
| Detection | Nebuliser Gas flow 3 L/min |
| | Drying gas flow 15 L/min |
| | Desolvation line temperature |
| | 250° C. Heat block temperature |
| | 400° C. Collision energy −35.0 |
| | MRM 233.2 > 174.20; 233.2 > 216.20 |
| Duration of analysis | 25 minutes |
| Injection volume | 1 μL |
| Standard preparation: | |

```
  1. Accurately weigh 1 mg of Melatonin reference standard into a 100 mL volumetric flask.
  2. Add a portion of 5% Methanol and sonicate for 10 minutes.
  3. Cool solution to room temperature.
  4. Make up to volume with 5% Methanol.
  5. Mix well.
  6. Filter through 0.45 μm nylon filter prior to injection.
```

Sample preparation:

```
  1. Accurately weigh 100 mg of sample extract into a 100 mL volumetric flask.
  2. Add a portion of 5% Methanol and sonicate for 30 minutes.
  3. Cool solution to room temperature.
  4. Make up to volume with 5% Methanol.
  5. Mix well.
  6. Filter through 0.45 μm nylon filter prior to injection.
```

System Suitability:

Inject the working standard solution six times and calculate the mean area and the RSD. The
RSD should not be more than 5% for the peak of Melatonin.

Calculation:

$\begin{matrix}Content of Melatonin \\ ( \% , \text{w/w} )\end{matrix} = \frac{A spl}{A std} \times \frac{C std}{C spl} \times Std purity \times 100$

| Where: | A spl | = peak area of Melatonin from sample solution |
|---|---|---|
| | A std | = peak area of Melatonin from standard solution |
| | C spl | = concentration of the sample solution in mg/mL |
| | C std | = concentration of the standard solution in mg/mL |

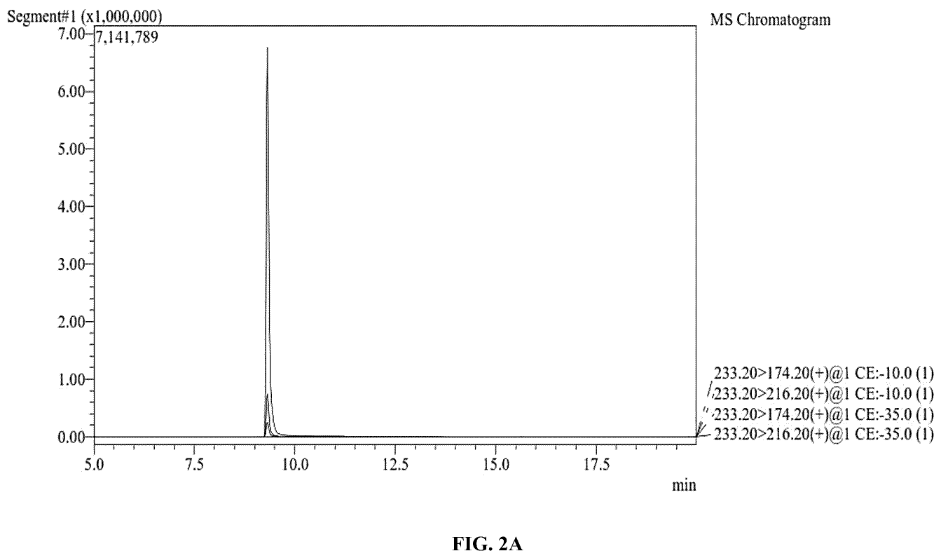

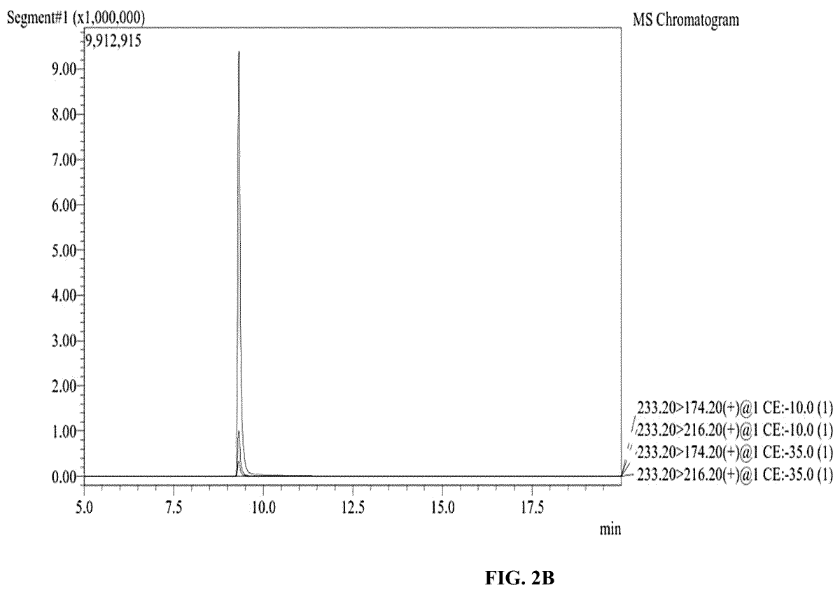

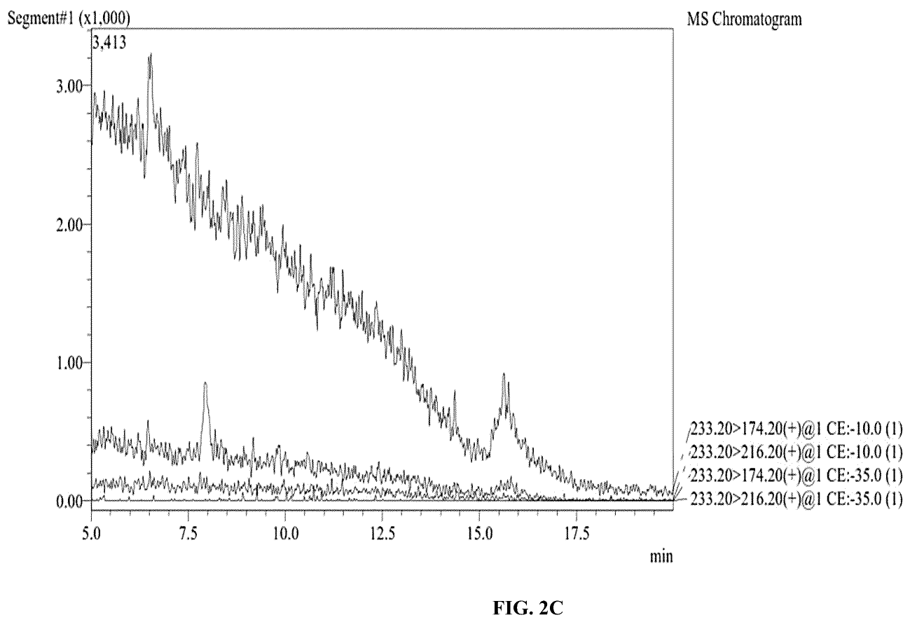

FIG. 2A is a graph showing an example chromatogram of standard; FIG. 2B is a graph showing an example chromatogram of sample; and FIG. 2C is a graph showing an example chromatogram of blank.

Since the melatonin extract and/or herbal matrices are biologically active on MT1 and 5 MT2 receptors in terms of binding and signal transduction. The melatonin extract increases the activity of the endogenous melatonin as well as increasing exogeneous melatonin, thus being a synergistic effect on treating sleep conditions. In one embodiment, the treatment of a sleep condition comprising administering low amounts of the melatonin extract in the amounts of about 0.03 g to about 0.5 g of the melatonin extract, which is equivalent to about 300 mcg to about 5 mg of natural melatonin as to trigger a physiological effect in humans.

### Example 2: The Efficacy of the Melatonin Extract Compared to a Comparator for Melatonin Absorption and a Pilot for Sleep Efficacy-a Randomized, Double-Blind, Cross-Over Study

The aim of this study is to assess the efficacy of a natural, food derived melatonin (Prosomnial™) gummy prepared according to the steps in FIG. 1B above compared to a synthetic melatonin gummy for melatonin absorption and sleep quality. The hypothesis is that a natural, food derived melatonin prepared by the steps above and a comparable synthetic melatonin will be equivalent in their absorption and impact on sleep quality.

### Investigational Product

TABLE 1b
Investigational Products (IP).

| Product | Intervention | Content per gummy per day |
|---|---|---|
| 1 | Active | Prosomnial gummy 100 mg equivalent to |
| | | melatonin 1 mg |
| 2 | Comparator | Synthetic melatonin gummy 1 mg |

Participants consumed 1 gummy at each PK visit (visits 2 and 3), and 1 gummy per night for 7 nights of each trial product. Each trial product consumption was separated by a 2-week washout period.

### Methods

Participants: Twenty-seven participants meeting the screening criteria detailed below were enrolled in the trial from Brisbane, Queensland.

Inclusion Criteria: Adults aged 18 years or older; Generally healthy; BMI between 18.5 and 29.9 kg/m²; Able to provide informed consent; Experience difficulty initiating sleep (>20 minutes); Agree to refrain from participation in other clinical trials while enrolled; Agree not to alter current diet, exercise frequency, or intensity throughout the study; Agree to avoid medicines, supplements, or foods for sleep, stress, or anxiety, except for the test product during the study; Females using a prescribed form of birth control (e.g., oral contraceptive).

Exclusion Criteria: Use of prescription, over-the-counter medications, or herbal supplements for sleep, stress, or anxiety; Use of CBD or aromatherapy for managing sleep, stress, or anxiety; Diagnosed chronic sleep disorders (e.g., insomnia, restless leg syndrome, sleep apnea); Serious illnesses* (e.g., mood disorders such as depression, anxiety, or bipolar disorder; neurological disorders such as MS; kidney, liver, or heart conditions); Unstable illnesses** (e.g., diabetes, thyroid dysfunction); Current malignancy (excluding Basal Cell Carcinoma) or treatment with chemotherapy/radiotherapy in the past 2 years; Use of anticoagulants (e.g., Coumadin/Warfarin, Heparin, Dalteparin, Enoxaparin, or low-dose aspirin); Active smokers, nicotine users, or individuals with drug (prescription or illicit) abuse; Chronic past or current alcohol use (>14 alcoholic drinks per week); Pregnant or lactating women; Allergies to any ingredients in the active or comparator formula; Current or recent (within the past month) participation in another clinical trial; Any condition deemed unsuitable for participation by the investigator *Serious illnesses are those that pose risks to mortality, impact quality of life, or cause significant symptoms or treatment burden.**Unstable illnesses are conditions not managed with stable medication doses or with fluctuating severity.

Recruitment and Screening Potential participants were informed at screening of their ineligibility if they failed to meet the inclusion/exclusion criteria. Interested individuals reviewed the Study Summary Page and completed a brief eligibility questionnaire. Those deemed eligible were provided with an electronic copy of the Participant Information and Consent Form (PlCF) for review.

At least 24 hours after receiving the PlCF, participants attended a telehealth consultation with a trial coordinator. During this session: Participants were screened against the inclusion/exclusion criteria; A full explanation of the trial procedures and requirements was provided; and Participants were encouraged to ask questions, request clarification, and consult family or friends before providing consent.

Eligible participants who agreed to participate signed an electronic consent form to confirm their understanding and willingness to enroll.

Trial Procedures Participants who passed screening were enrolled and assigned an investigational product (IP) in identical containers labelled with a unique product number (per randomization code). Participants remained blinded to their allocated product.

Study Timeline Participants attended the RDC Clinic three times during the study period:
1. Screening Phase (Remote): Telehealth screening and informed consent
2. Visit 1: Screening and Consent: Conducted within 4 weeks prior to trial initiation
3. Baseline 1:3-day sleep recording prior to Visit 2
4. Visit 2: PK 1

Participants attended in a fasted state (no food after 10 PM the previous night).

A cannula was inserted into the antecubital vein, and baseline blood samples were collected.

Participants consumed their allocated IP with 250 ml of water.

Blood samples were collected at: 10, 20, 30, 45, 60, 75, 90, 120, 150 minutes, and 3-, 4-, 6-, and 8-hours post-ingestion.

Cannulas were removed after all samples were collected.

Meals were provided: Breakfast: Post-IP consumption; Lunch: Post 4-hour blood sample; and Snacks: Afternoon (all food intake recorded by the trial coordinator). Water consumption was unrestricted (ad libitum).
5. Supplementation Phase 1:7-day supplementation at home with the allocated IP
6. Washout Period: 2-weeks between supplementation phases
7. Baseline 2:3-day sleep recording prior to Visit 3
8. Visit 3: PK 2: Procedures identical to Visit 2
9. Supplementation Phase 2:7-day supplementation at home with allocated IP.
10. Final Interview: Conducted via phone.

Participant Requirements: Participants completed a 3-day baseline sleep diary before both Visit 2 and Visit 3 to establish stable sleep patterns. Daily online sleep diaries were completed throughout each 7-day supplementation period. Unused IP from Visit 2 was returned at Visit 3. Unused IP from Visit 3 was disposed of at the study's conclusion.

Blood Sampling and Measurements During PK visits, blood samples were collected over 8 hours as outlined above. The cannula was removed after the final sample, and participants were monitored throughout. All meals and water intake were controlled and recorded during PK visits.

A stable 3-day sleep recording period before each PK visit ensured baseline sleep patterns were captured for comparison during supplementation phases.

TABLE 2
Schedule of Activities

| | +/−3 days | | | | | | | |
|---|---|---|---|---|---|---|---|---|
| | | | | | | | | Final |
| Screening/ | Baseline | PK | Supplementation | | Baseline | PK | Supplementation | (phone |
| Enrolment | 1 | 1 | 1 | Washout | 2 | 2 | 2 | call) |
| Visit | | | | | | | | |
| 1 | — | 2 | — | — | — | 3 | — | 4 |

| | Number of Days | | | | | | | |
|---|---|---|---|---|---|---|---|---|
| Activity | 1-28 | 3 | 1 | 7 | 14 | 3 | 1 | 7 |

| | | | | | | | | |
|---|---|---|---|---|---|---|---|---|
| Information session and informed consent | X | | | | | | | |
| Assess eligibility via inclusion and exclusion | X | | | | | | | |
| criteria | | | | | | | | |
| Anthropometric measures - height, weight, | X¹ | | X² | | | | | |
| BMI, waist and hip circumference | | | | | | | | |
| Vital signs³ - blood pressure, pulse rate, O₂ | X | | X | | | X | | |
| saturation | | | | | | | | |
| Blood sample collection - 14 timepoints over | | | X | | | X | | |
| 8 hours | | | | | | | | |
| Consensus Sleep Diary (CSD-M) | | X | X | X | X | X | X | |
| IP supplementation | | | X | X | | X | X | |
| IP compliance monitoring | | | X | X | | X | X | X |

| | |
|---|---|
| Adverse event monitoring | As occurs |
| Concomitant medication monitoring | As occurs |

¹self-reported height and weight during phone screening to be confirmed at Visit 1,
²excluding height,
³after sitting for 5 minutes

### Outcome Measures

Primary outcomes: Total absorption of melatonin as measured by AUC0-8 via blood sample.

Secondary outcomes: Cₘₐₓ via blood sample, Tₘₐₓ via blood sample, T½ via blood sample, Change in sleep quality as assessed by sleep diary, Change in sleep onset time as assessed by Consensus Sleep Diary (CSD-M), Change in sleep disturbance as assessed by sleep diary, and Number and severity of adverse events via AE monitoring.

### Results

### Demographics

Twenty-six participants were enrolled in this study, with twenty-four participants completing the PK portion for product 1 (Prosomnial) and twenty-three completing product 2 (Comparator). Of the participants completing the PK portion, 21 completed the 7-day sleep diary for both product 1 (Prosomnial) and product 2 (comparator).

As this was a cross-over study design, there was no significant difference between group characteristics at any point (Table 3 and Table 4).

TABLE 3
Participant demographics.

| | Baseline |
|---|---|
| Height (m) | 1.70 ± 0.06 |
| Weight (kg) | 72.8 ± 11.5 |
| BMI (kg/m2) | 25.2 ± 3.0 |
| Waist circumference (cm) | 84.2 ± 8.5 |
| Hip circumference (cm) | 100.2 ± 6.9 |

TABLE 4
Participant vitals at each visit.

| | Prosomnial | Comparator |
|---|---|---|
| Systolic BP (mmHg) | 118.8 ± 12.3 | 113.8 ± 11.9 |
| Diastolic BP (mmHg) | 74.1 ± 10.2 | 72.6 ± 8.7 |
| Pulse (BPM) | 73.2 ± 8.1 | 70.4 ± 8.8 |
| O2 saturation (%) | 97.4 ± 1.3 | 98.3 ± 0.8 |

### Plasma Melatonin

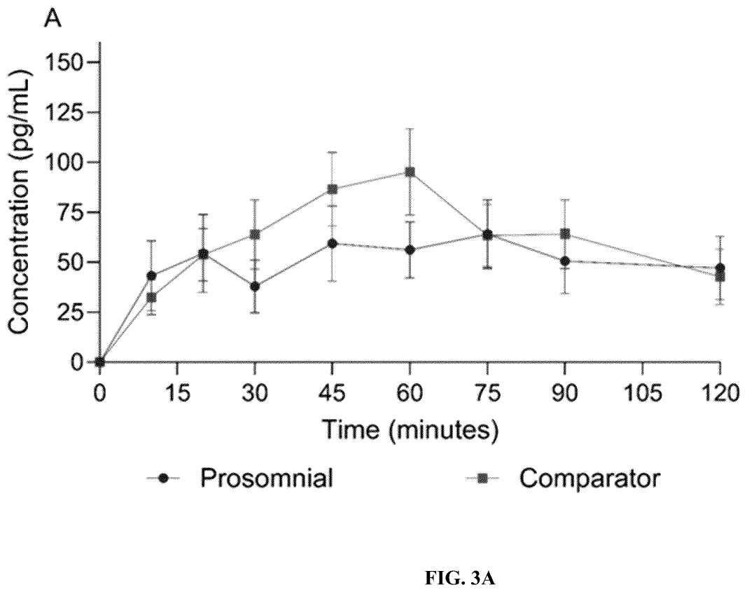

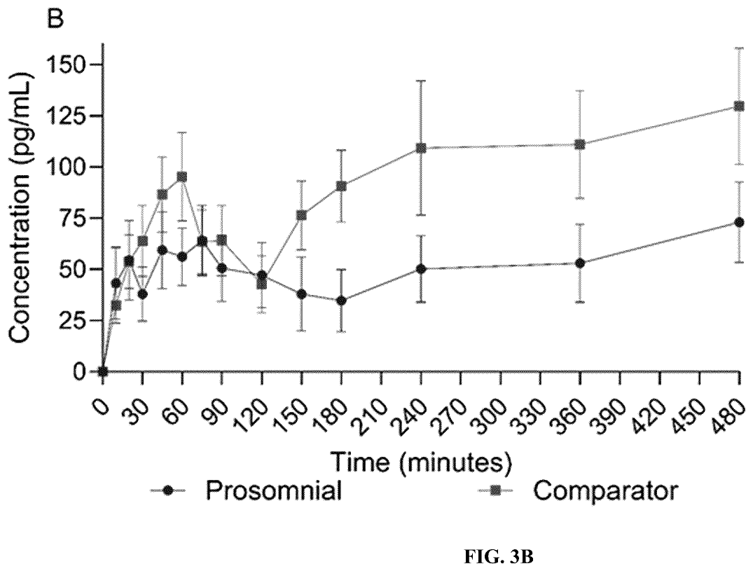

When the first 2-hours of absorption were assessed, there was no significant difference between groups (p=0.4, FIG. 3B).

TABLE 6
Melatonin absorption data over 2-hours. Values calculated from each
participants specific AUC, Tₘₐₓ and Cₘₐₓ. Data presented is mean.

| | Prosomnial | Comparator | p-Value |
|---|---|---|---|
| AUC₀₋₈ (pg · h/mL) | 5,994 | 7,425 | 0.39 |
| Tₘₐₓ (min) | 75 | 60 | N/S |
| Cₘₐₓ (pg/mL) | 64.02 | 95.28 | N/S |

### Sleep Diary

There was no significant difference between groups baselines (Table 7). When relative change from baseline for each group was factored, there was no significant difference between groups for any measure. The Prosomnial group had a significant change in sleep latency, total sleep time, sleep quality and feeling rested compared to baseline, where the comparator Melatonin had significant change in awakenings and feeling rested compared to their baseline.

TABLE 7
Sleep diary data.

| | Prosomnial | Prosomnial | Comparator | Comparator |
|---|---|---|---|---|
| | Baseline | After 7 days | Baseline | After 7 days |
| How long did it take you to fall | 38.0 ± 22.0 | 28.3 ± 23.4* | 34.8 ± 20.8 | 28.3 ± 50.6 |
| asleep? (mins) | | | | |
| How many times did you wake | 1.8 ± 0.9 | 1.4 ± 1.0 | 1.8 ± 1.1 | 1.2 ± 0.6* |
| up, not counting final | | | | |
| awakening? | | | | |
| In total, How long did these | 27.1 ± 27.6 | 26.9 ± 29.2 | 37.6 ± 34.7 | 25.3 ± 21.4 |
| awakenings last? (mins) | | | | |
| In total, How long did you | 380.9 ± 65.7 | 405.5 ± 46.8* | 378.9 ± 64.8 | 404.8 ± 74.3 |
| sleep? (mins) | | | | |
| How would you rate the | 3.0 ± 0.8 | 3.4 ± 0.6* | 3.0 ± 0.7 | 3.4 ± 0.7 |
| quality of your sleep? | | | | |
| How rested or refreshed did | 2.7 ± 0.7 | 3.0 ± 0.6* | 2.6 ± 0.8 | 3.1 ± 0.9* |
| you feel when you woke-up for | | | | |
| the day? | | | | |
| How many times did you nap | 0.3 ± 0.4 | 0.2 ± 0.4 | 0.3 ± 0.4 | 0.3 ± 0.3 |
| or doze? | | | | |
| In total, how long did you nap | 25.8 ± 25.4 | 21.5 ± 79.7 | 34.6 ± 47.8 | 20.4 ± 25.8 |
| or doze? (mins) | | | | |

*significant difference from baseline

In summary, 100 mg of Prosomnial™ including 1 mg of natural, plant-derived melatonin delivered in gummy/soft pastille format demonstrated statistically significant improvements over baseline in the areas of sleep latency (time taken to fall asleep), total sleep time, sleep quality, and of feeling rested and refreshed.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method for preparing a melatonin-enriched extract of Pistacia vera (Pistachio) seed/kernel comprising: supercritical fluid to remove fatty acids and oily fraction from seed/kernel from a melatonin extract; washing the melatonin extract with water; fermenting the milled seed/kernel using a yeast/culture medium; extracting melatonin with a plurality of solvents; repeated extracting until in-process testing confirms melatonin assay has met an acceptance criteria; step of concentrating the liquid extract solution.

2. The method of claim 1, wherein the plurality of solvents are selected from the group consisting of: ethanol, methanol, water, acetone, hexane, butanol, and/or other suitable solvents.

3. The method of claim 2, wherein the supercritical fluid extracting is by CO₂.

4. A method for preparing a melatonin-enriched extract of Pistacia vera (Pistachio) seed/kernel comprising: selecting crude raw material Pistacia vera (Pistachio) seed/kernel with endogenous natural melatonin content between about 0.02 and about 0.04%; crushing of an outer shell of the Pistachio and removing the outer shell; supercritical fluid extracting to remove fatty acids and oily fraction from seed/kernel; washing the melatonin extract with water; extracting melatonin with a plurality of solvents; repeated extracting until in-process testing confirms melatonin assay has met an acceptance criteria; concentrating the liquid extract solution; drying by a suitable method; standardizing to contain at least 1% melatonin in a final composition; and applying quality control measures include herbal identification, assay for melatonin potency, radiocarbon testing to ensure avoidance of synthetic additives, and general contaminant testing.

5. The method of claim 4, wherein the quality control measures include solvent residues, pesticide residues, aflatoxins, microbiology, heavy metals, pyrrolizidine alkaloids, polycyclic aromatic hydrocarbons.

6. The method of claim 5, wherein the standardizing step is achieved by sub-lot blending or through the use of an appropriate carrier/excipient.

7. A method of increasing sleeping efficacy in a subject, comprising: administering an effective amount of a melatonin extract from Pistachio; and increasing sleeping efficacy by the melatonin extract from Pistachio in the subject compared to a synthetic melatonin extract.

8. The method of claim 7, further comprising administering an effective amount of herbal matrices including arginine, lutein, resveratrol, and Tryptophan.

9. The method of claim 8, wherein the melatonin extract comprises 1 mg in a gummy format; administering the melatonin extract for a period of 7 days; measuring a sleep baseline without administration of the melatonin extract; and the melatonin extract demonstrating statistically significant improvements over baseline in the areas of sleep latency, total sleep time, sleep quality, and of feeling rested and refreshed; wherein sleep latency is the time taken to fall asleep.
